(19) United States
(12) Patent Application Publication
(10) Pub. No.: US 2004/0170692 A1
(43) Pub. Date: Sep. 2, 2004

(54) Calcium phosphate bodies and a process for making calcium phosphate bodies

(75) Inventors: Delbert E. Day, Rolla, MO (US); Xue Han, Baltimore, MD (US); Kenan Patrick Fears, Anderson, SC (US)

Correspondence Address: SENNIGER POWERS LEAVITT AND ROEDEL, ONE METROPOLITAN SQUARE, 16TH FLOOR, ST LOUIS, MO, 63102, US

(73) Assignee: The Curators of the University of Missouri

(21) Appl. No.: 10/777,295

(22) Filed: Feb. 12, 2004

Related U.S. Application Data
(60) Provisional application No. 60/454,534, filed on Mar. 12, 2003.
(60) Provisional application No. 60/450,941, filed on Feb. 26, 2003.
(60) Provisional application No. 60/446,926, filed on Feb. 12, 2003.

(51) Int. Cl. A61K 33/42; A61K 9/14

(52) U.S. Cl. 424/489; 424/602

(57) ABSTRACT

Among the various aspects of the present invention is a process for making calcium phosphate bodies comprising amorphous calcium phosphate, hydroxyapatite or calcium triphosphate, the bodies themselves and the use of such bodies in any of a variety of applications.

### REFERENCE TO RELATED APPLICATIONS

This application is a non-provisional application claiming priority from the following U.S. provisional applications: Serial No. 60/454,534, filed Mar. 12, 2003; Serial No. 60/450,941, filed Feb. 26, 2003, and Serial No. 60/446,926 filed Feb. 12, 2003, each of which is incorporated herein by reference in its entirety.

### BACKGROUND OF THE INVENTION

The present invention relates to calcium phosphate bodies and methods of preparing and using the same.

Calcium phosphate bodies and methods for their preparation and use have been disclosed. In general, they differ in the physical characteristics of the phosphate bodies and the method of chemical transformation of the starting glass to the calcium phosphate body. For example, Luo et al. (U.S. Pat. No. 5,858,318 ) describe the formation of spherical hydroxyapatite (a form of calcium phosphate) particles by spray drying and spray pyrolysis. Yasuikawa et al. (Materials Research Bulletin, 4, 589 (1999)) describe the preparation of spherical calcium phosphate bodies by transformation of a spherical precursor, calcium chelate decomposition, and precipitation using urea and cetyltrimethylammonium chloride. Paul et al. (J. Mater. Sci.: Mater. Med., 10, 383 (1999)) describe mixing hydroxyapatite in oils with a stabilizer and adding an agent to harden the hydroxyapatite spheres that form.

Starling et al.(U.S. Pat. No. 6,210,715) describe the preparation of hollow spheres of calcium phosphate by (1) a sol gel method or (2) a coating method. The sol gel method involves a calcium phosphate precipitated solution nozzle-sprayed onto an oleyl alcohol condensing solution. Alternatively, the coating method involves slurries of powders of calcium phosphate applied to the surfaces of wax or other organic microbeads and subsequent microbead removal by thermal decomposition or solvent extraction.

### SUMMARY OF THE INVENTION

Among the various aspects of the present invention is a process for making calcium phosphate bodies comprising amorphous calcium phosphate, hydroxyapatite, or calcium triphosphate, the bodies themselves and the use of such bodies in any of a variety of applications. In general, these bodies are prepared from a water-soluble glass body containing calcium.

Briefly, therefore, one aspect of the present invention is process for the preparation of a calcium phosphate body, the process comprising contacting a water-soluble glass body in the form of a sphere, fiber, flake or ellipsoid and a phosphate solution wherein the water-soluble glass body contains about 1 to about 40 wt. % CaO, about 5 to about 65 wt. % alkali metal oxide and about 20 to about 94 wt. % of a glass former.

Another aspect of the present invention is the bodies prepared from the above process.

A further aspect of the present invention is various methods of use of the calcium phosphate bodies. In one such method, a fluid delivery system is produced by (i) heat treating a calcium phosphate body at a temperature between about 90° C. and about 900° C., (ii) filling the calcium phosphate body with a desired fluid and (iii) administering the calcium phosphate body filled with a fluid to a subject. In another such method, a chemical species is separated from a fluid containing the species by affinity chromatography comprising (i) contacting the fluid with a macroscopically smooth calcium phosphate body to adsorb the chemical species onto the calcium phosphate body, (ii) separating the fluid from the calcium phosphate body and (iii) desorbing the species from the calcium phosphate body. In another such method, a calcium phosphate body is used as a bone substitute comprising administering the calcium phosphate body to a subject. In another such method, a calcium phosphate body is used as a diagnostic imaging agent comprising (i) filling the calcium phosphate body with a gas, (ii) heat treating the calcium phosphate body and (iii) administering the gas filled calcium phosphate body to a subject.

Other aspects and objects of the present invention will be, in part, apparent and, in part, pointed out hereinafter.

### DESCRIPTION OF THE DRAWINGS

FIG. 1 shows a schematic depiction of the transformation process that the water-soluble glass bodies undergo when immersed in or otherwise contacting a phosphate solution.

FIG. 2 is a series of scanning electron microscope images which show calcium phosphate microspheres prepared as described in Example 1. FIGS. 2(a) to 2(c) show calcium phosphate microspheres made from water-soluble glass bodies with a composition of 15 wt. % CaO, 10.7 wt. % Li₂O, 74.3 wt. % B₂O₃.

FIG. 3 is a scanning electron microscope image which shows a cross section of a hollow calcium phosphate fiber as described in Example 2.

FIG. 4 is a scanning electron microscope image showing the appearance of the cross section of an agglomerate of calcium phosphate bodies prepared as described in Example 3.

FIG. 5 is a series of scanning electron microscope images showing magnified images of the cross section of the wall of a calcium phosphate microsphere prepared as described in Example 1, then heat treated for one hour at (a) 300° C., (b) 500° C., (c) 600° C. and (d) 900° C.

FIG. 6 is a series of scanning electron microscope images showing a (a) hollow calcium phosphate sphere and (b) a magnification of the wall of the calcium phosphate sphere. The calcium phosphate sphere was prepared as described in Example 1 from a water-soluble glass sphere containing 25 wt. % CaO, 9.4 wt. % Li₂O and 65.6 wt. % B₂O₃.

FIG. 7 is a graph showing the release of bovine serum albumin (BSA) from hollow hydroxyapatite microspheres as described in Example 4.

FIG. 8 is a HPLC chromatograms illustrating the separation of BSA and lysozyme using the hydroxyapatite microspheres as the column packing material as described in Example 5.

FIG. 9 is a HPLC chromatograms illustrating the separation of BSA, myoglobin and lysozyme using the hydroxyapatite microspheres as the column packing material as described in Example 5.

### DETAILED DESCRIPTION

In general, the calcium phosphate bodies of the present invention are derived from a water-soluble glass body containing calcium. When the glass is immersed in or otherwise contacted with an aqueous phosphate solution, the glass dissolves, thereby releasing Ca²⁺ ions into the aqueous phosphate solution. In this solution, Ca²⁺ ions react with PO₄³⁻ and OH⁻ ions to form calcium phosphate which has a relatively low solubility limit in the aqueous phosphate solution. As the dissolution of the glass proceeds, the concentration of calcium phosphate increases in the solution until the solubility limit of calcium phosphate is exceeded and, as a consequence, calcium phosphate is deposited as a porous calcium phosphate layer on the outer surface of the water-soluble glass body. The formation of this porous calcium phosphate layer on the water-soluble glass body, however, does not prevent further dissolution of the water-soluble glass. Rather, the glass continues to dissolve and, as it does, the thickness of the porous calcium phosphate layer increases. Eventually, the water-soluble glass is completely dissolved, leaving only a porous calcium phosphate body.

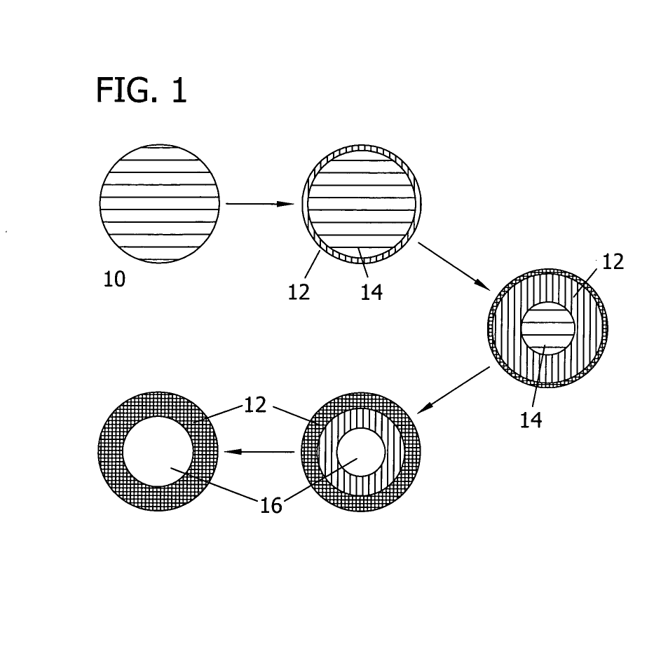

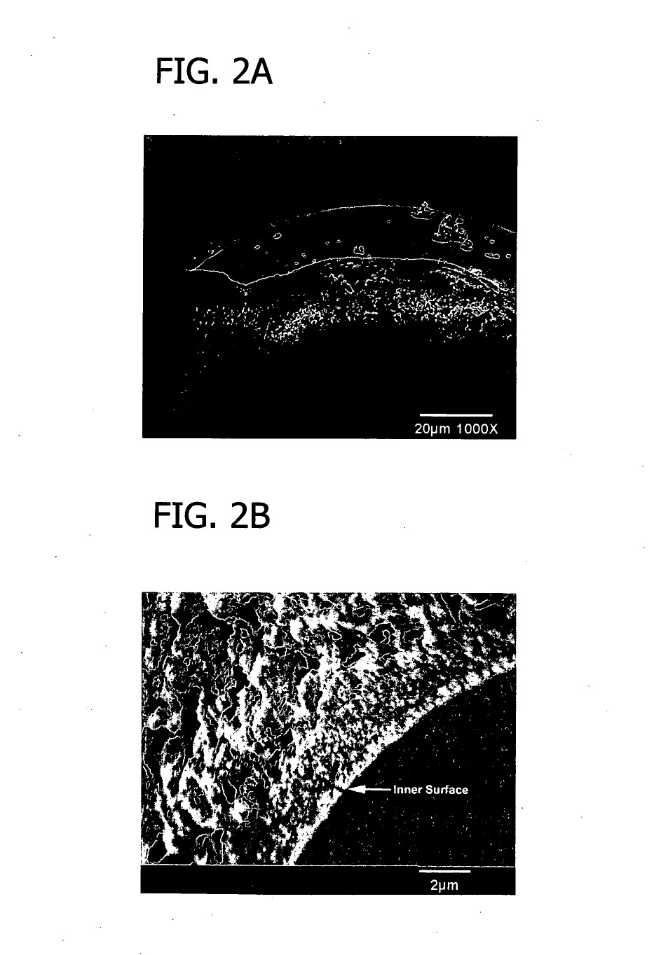

Referring now to FIG. 1, one embodiment of the transformation of a water-soluble glass body into a calcium phosphate body of the present invention is schematically illustrated. To begin the process, a body 10 which initially comprises a water-soluble glass containing calcium is immersed in an aqueous phosphate solution (not shown). The water-soluble glass begins to dissolve in the aqueous phosphate solution, thereby releasing Ca²⁺ and other ions into the aqueous phosphate solution. In the aqueous phosphate solution, the Ca²⁺ ions react with phosphate and hydroxide ions to form calcium phosphate which, in turn, deposits to form a porous calcium phosphate layer 12 over a water-soluble glass core 14. As time passes and the dissolution of the water-soluble glass proceeds, the diameter of the water-soluble glass core 14 decreases and the thickness of the outer porous calcium phosphate layer 12 increases. In addition, the porous calcium phosphate layer 12 may originally appear to be predominantly, if not entirely, amorphous calcium phosphate; as time passes, however, the amorphous calcium phosphate is transformed into needle-shaped crystals of hydroxyapatite, a crystalline form of calcium phosphate having the formula Ca₅(PO₄)₃OH which packs poorly and is, as a result, relatively porous. If sufficient time is allowed to pass with the body 10 immersed in the aqueous phosphate solution, the water-soluble glass core 14 is completely dissolved, leaving only the porous calcium phosphate layer 12 which defines a shell surrounding an inner void 16. In addition, given sufficient time, the needle-shaped crystals of hydroxyapatite may grow into plate-shaped crystals.

The water-soluble glass starting material comprises calcium and a balance of elements which enable the glass to dissolve in an aqueous phosphate solution. Preferably, the composition of the glass is selected to enable the glass to dissolve at a commercially practical rate in the aqueous phosphate solution. For example, it is generally preferred that the glass have a solubility of at least about 0.1 mol/L in water at 37° C. This aqueous solution may be a mixture of water with any miscible solvent. Exemplary solvents that are miscible with water are methanol, ethanol, isopropanol, acetone, ethers and the like. Stated another way, the water-soluble glass dissolves in an aqueous solvent system at 37° C. within about 1 hour to 2 weeks, about 1 hour to 1 week, about 4 hours to 3 days, or preferably, within about 4 hours to 24 hours. It will be understood that the dissolution time of the water-soluble glasses will be dependent on the size of the water-soluble glass body and the starting glass composition. For example, water-soluble glass bodies that are smaller in size will dissolve more rapidly as compared to a water-soluble glass body of a larger size.

In general, glasses having a commercially practical solubility have a glass composition containing (i) a calcium component, comprising CaO, CaF₂, or mixtures thereof, (ii) an alkali metal oxide component, comprising an alkali metal oxide or mixtures of alkali metal oxides, and (iii) one or more glass formers. In one embodiment, the water-soluble glass contains about 1-40 wt. % of the calcium component, about 5-65 wt. % of the alkali metal oxide component and about 20-94 wt. % of the glass former(s). In another embodiment, the water-soluble glass contains a calcium component, an alkali metal oxide component and one or more glass formers as specified, provided, however, the glass does not contain 20-35% Na₂O, 20-35% CaO, 0-10% P₂O₅, and 30-50% B₂O₃.

In one embodiment, the glass contains about 5 to 40 wt. % of CaO, CaF₂, or mixtures thereof. In a second embodiment, the glass contains about 10 to 30 wt. % of CaO, CaF₂or mixtures thereof. In a third embodiment, the glass contains about 10 to 15 wt. % of CaO, CaF₂ or mixtures thereof. In a fourth embodiment, the glass contains about 15 wt. % of CaO, CaF₂ or mixtures thereof. In each of these embodiments, it is contemplated that CaF₂ may be omitted as a constituent of the glass.

The alkali metal oxide component may be, for example, Li₂O, Na₂O, K₂O, Rb₂O, Cs₂O, or a mixture thereof. In one embodiment, the glass contains about 8 wt. % to about 55 wt. % of Li₂O, Na₂O, K₂O, Rb₂O, Cs₂O, or a mixture thereof. In another embodiment, the glass contains about 10 to about 52 wt. % of Li₂O, Na₂O, K₂O, Rb₂O, Cs₂O, or a mixture thereof. Typically, the glass contains about 5 to about 16 wt. % of Li₂O.

The glass former may be, for example, SiO2, P₂O₅, B₂O₃, GeO₂ or a mixture thereof. In one embodiment, the glass former is SiO₂, P₂O₅, B₂O₃ or a mixture thereof constituting about 20 to about 94 wt. % of the glass composition. In another embodiment, the glass former is SiO₂, B₂O₃ or a mixture thereof constituting about 20 to about 94 wt. % of the glass composition. In yet a further embodiment, the glass former is B₂O₃ constituting about 20 to about 94 wt. % of the glass composition. In another embodiment, the glass former is B₂O₃ constituting about 42 to about 84 wt. % of the glass composition. Typically, the glass former is B₂O₃ constituting about 57 to about 82 wt. % of the glass composition.

As previously noted, the water-soluble glass will typically contain about 1 to 40 wt. % of a calcium component, about 5 to 65 wt. % of an alkali metal oxide component and about 20 to 94 wt. % of the glass former. In a first embodiment, the glass contains CaO and an alkali metal oxide component, which is Li₂O, Na₂O, K₂O or a mixture thereof; for example, in this embodiment, the glass formed is a glass containing 5 to 40 wt. % of CaO and 5 to 40 wt. % of the alkali metal oxide component, a glass containing 10 to 30 wt. % of CaO and 6 to 35 wt. % of the alkali metal oxide component, or a glass containing 10 to 15 wt. % of CaO and 8 to 35 wt. % of the alkali metal oxide component. In a second embodiment, the glass contains CaO and the alkali metal oxide component is Li₂O, Na₂O or a mixture thereof; for example, in this embodiment, the glass formed is a glass containing 5 to 40 wt. % of CaO and 5 to 30 wt. % of the alkali metal oxide component, a glass containing 10 to 30 wt. % of CaO and 6 to 28 wt. % of the alkali metal oxide component, or a glass containing 10 to 15 wt. % of CaO and 8 to 28 wt. % of the alkali metal oxide component. In a third embodiment, the glass contains CaO and the alkali metal oxide is Li₂O; for example, in this embodiment, the glass formed is a glass containing 5 to 40 wt. % of CaO and 5 to 17 wt. % of the alkali metal oxide, a glass containing 10 to 30 wt. % of CaO and 6 to 16 wt. % of the alkali metal oxide, or a glass containing 10 to 15 wt. % of CaO and 8 to 15 wt. % of the alkali metal oxide. In a fourth embodiment, the glass contains CaO, the alkali metal oxide component is Li₂O, Na₂O, K₂O or a mixture thereof and the glass former is B₂O₃; for example, in this embodiment, the glass formed is a glass containing 5 to 40 wt. % of CaO, 5 to 40 wt. % of the alkali metal oxide component and 20 to 90 wt. % of B₂O₃, a glass containing 10 to 30 wt. % of CaO, 6 to 35 wt. % of the alkali metal oxide component and 35 to 84 wt. % of B₂O₃, or a glass containing 10 to 15 wt. % of CaO, 8 to 35 wt. % of the alkali metal oxide component and 50 to 82 wt. % of B₂O₃. In a fifth embodiment, the glass contains CaO, the alkali metal oxide component is Li₂O, Na₂O or a mixture thereof and the glass former is B₂O₃; for example, in this embodiment, the glass formed is a glass containing 5 to 40 wt. % of CaO, 5 to 30 wt. % of the alkali metal oxide component and 30 to 90 wt. % of B₂O₃, a glass containing 10 to 30 wt. % of CaO, 6 to 28 wt. % of the alkali metal oxide component and 42 to 84 wt. % of B₂O₃, or a glass containing 10 to 15 wt. % of CaO, 8 to 28 wt. % of the alkali metal oxide component and 57 to 82 wt. % of B₂O₃. In a sixth embodiment, the glass contains CaO, the alkali metal oxide is Li₂O and the glass former is B₂O₃; for example, in this embodiment, the glass formed is a glass containing 5 to 40 wt. % of CaO, 5 to 17 wt. % of the alkali metal oxide and 43 to 90 wt. % of B₂O₃, a glass containing 10 to 30 wt. % of CaO, 6 to 16 wt. % of the alkali metal oxide and 54 to 84 wt. % of B₂O₃, or a glass containing 10 to 15 wt. % of CaO, 8 to 15 wt. % of the alkali metal oxide and 70 to 82 wt. % of B₂O₃. In yet another embodiment, the glass contains CaO, an alkali metal oxide component of Li₂O, Na₂O, K₂O, Rb₂O, Cs₂O or a mixture thereof and the glass former is B₂O₃. In still another embodiment, preferably, the alkali metal oxide component is Li₂O, Na₂O, K₂O, Rb₂O, Cs₂O or a mixture thereof and the glass former is B₂O₃, where the molar ratio of the B₂O₃ to alkali metal oxide component ranges from about 2 to 1 to about 4 to 1 or is about 3 to 1.

In one embodiment, the water-soluble glass body contains a dopant. In general, doped water-soluble glass bodies have (i) cations substituted for the calcium in the glass and/or (ii) anions substituted for the glass former. The glass bodies may contain up to about 10 wt. % of the dopant material. In addition, the calcium phosphate bodies may be doped by doping the phosphate solution with (i) cations to replace a portion of the calcium ions in the calcium phosphate body and/or (ii) anions to replace a portion of the phosphate ions in the calcium phosphate body. The calcium phosphate body may contain a concentration of dopant cations or anions that would result in up to about 10 wt. % of the dopant cations or anions incorporated into the resulting calcium phosphate body. The lower limit of the dopant anion or cation may be a trace; this trace may be about 0.01 wt. % or as small an amount as can be weighed out or measured by volume.

The water-soluble glass may optionally be doped with other cations in addition to calcium to produce calcium phosphate bodies where other cations are substituted for calcium ions. For example, cation-substituted water-soluble glass bodies may be prepared by adding the desired metal oxide to the mixture of (i) calcium carbonates, calcium sulfates, or calcium nitrate, (ii) alkali metal carbonates, alkali metal sulfates, or alkali metal nitrates, and (iii) glass formers to form the desired cation doped water-soluble glass body. Exemplary metal oxides that may be used as dopants are Cr₂O₃, Al₂O₃, CuO, Cu₂O, MgO, SrO, BaO, FeO, Fe₂O₃, Bi₂O₃, ZnO, MnO, Mn₂O₃, NiO, Y₂O₃, ZrO₂, TiO₂, rare earth oxides, or combinations thereof; with the dopant metal oxide being substituted for the glass former when preparing the water-soluble glass.

Alternatively or in addition, the calcium phosphate bodies produced in the above described transformation process may optionally be doped with other ions in addition to phosphate by adding the appropriate ion to the aqueous phosphate solution. For example, the calcium phosphate bodies may be carbonated by adding a source of CO₃²⁻ ions, such as an alkali metal carbonate, to the phosphate solution or by bubbling carbon dioxide into the aqueous phosphate solution. Exemplary alkali metal carbonates include Li₂CO₃ Na₂CO₃, K₂CO₃, Rb₂CO₃, Cs₂CO₃ and combinations thereof. Moreover, the calcium phosphate bodies produced in the transformation process may be halogenated by adding a halide salt, such as an alkali metal halide, to the phosphate solution. Exemplary alkali metal halides are alkali metal fluorides, alkali metal chlorides, alkali metal bromides and alkali metal iodides, where the alkali metal is lithium, sodium, potassium, rubidium or cesium. In addition, a source of SO₄²⁻ ions may be added to the phosphate solution. Exemplary sources of the sulfate ion are alkali metal sulfates and all other soluble metal sulfates. Additionally, a source of NH₄⁺ ions may be added to the phosphate solution; an exemplary source of ammonium ions is ammonium phosphate. Other ions may be added to the phosphate solution in order to dope the product calcium phosphate body; ion identity is limited only by solubility in the phosphate solution and reactivity with the water-soluble glass body.

The water-soluble glass bodies described above are prepared by combining the desired amounts of carbonates, nitrates, sulfates, or the like of calcium and carbonates, nitrates, sulfates, or the like of alkali metals with glass formers. For example, CaCO₃₍ₛ₎, CaSO₄₍ₛ₎ or a combination of CaCO₃₍ₛ₎ and CaSO_{4(S)}, are combined with carbonates of lithium, sodium, potassium, rubidium and cesium and glass formers, SiO_{2(S)}, P₂O₅₍ₛ₎, GeO₂₍ₛ₎ or H₃BO_{3(S)}, and a melt is formed by conventional means. In water-soluble glasses where the calcium content is greater than 25 wt. %, a mixture of CaCO₃₍ₛ₎ and CaSO₄₍ₛ₎ is believed to reduce the crystallization tendency of the glass. While the carbonates of calcium and alkali metals are typically used, other salts of calcium and alkali metals such as nitrates, sulfates, or the like may be used.

Using this melt, the water-soluble glass may be molded (using, e.g., a graphite mold), drawn or otherwise formed into a desired shape; for example, the glass body may be in the form of a bar, rod, cube, ellipsoid or the like.

In one embodiment, the water-soluble glass bodies are smooth bodies. Smooth bodies are characterized as having no sharp edges or points; for example, sharp edges or points are characteristic of crushed or shattered glass. Exemplary smooth bodies are spheres, fibers, ellipsoids, and the like. The smooth water-soluble glass bodies may have a composition of 1-40 wt. % of a calcium component, about 5-65 wt. % of an alkali metal oxide component and about 20-94 wt. % of one or more glass formers. When the smooth water-soluble glass body is a sphere with a diameter greater than about 1 μm, in one embodiment the glass does not contain 20-35% Na₂O, 20-35% CaO, 0-10% P₂O₅, and 30-50% B₂O₃.

The water-soluble glass bodies may be transformed into a calcium phosphate body by immersing the water-soluble glass body in or otherwise contacting the water-soluble glass body with an aqueous phosphate solution. Typically, the solution will be about 0.001M to 1.0M in phosphate ions. In one embodiment, the phosphate concentration is, preferably, about 0.025M to about 0.5M; more preferably, about 0.25M. Although not necessarily preferred, the aqueous solution may additionally contain other solvents such as methanol, ethanol, isopropanol, acetone, ethers and the like, provided they are miscible with water and the solution is predominantly water (on a weight basis). In one embodiment, the phosphate solution is formed by dissolving an alkali metal hydrogen phosphate compound in water. For example, alkali metal hydrogen phosphate compounds suitable for the reaction are Li₂HPO₄, Na₂HPO₄, K₂HPO₄, Rb₂HPO₄, Cs₂HPO₄, and the like.

The aqueous phosphate solution will typically have a pH of about 7-10, preferably at about 9. The pH may be adjusted, for example, using standard strong acids and bases such as hydrochloric acid and sodium hydroxide.

The temperature of the aqueous phosphate solution and the time allowed for transformation of the water-soluble glass body to a calcium phosphate body is not narrowly critical and, as a practical matter, will be influenced by commercial considerations. Nevertheless, the temperature of the aqueous phosphate solution will typically be in the range of about 20-90° C., with temperatures in the range of about 25-50° C., or about 30-40° C.; or even about 37° C. being preferred for many applications. At temperatures within these ranges, the transformation to a calcium phosphate body will range from about 1 hour to about 2 weeks, about 4 hours to 3 days, or even about 4 hours to 24 hours. For example, in one embodiment of the present invention, the transformation of the water-soluble glass body into a calcium phosphate body is carried out in a solution having a pH of about 9, a phosphate ion concentration of about 0.25M, a temperature of about 37° C. and for a transformation period of about 24 hours.

The transformation process that produces the calcium phosphate bodies of the invention may take place in any reaction vessel appropriate to the temperature of the reaction and corrosive properties of the reactants. Upon completion of the reaction, the products are rinsed with a nonaqueous solvent and dried by conventional methods.

In one embodiment of the present invention, the dissolution process is allowed to go to completion; that is, until the water-soluble glass is completely dissolved. Alternatively, the dissolution process may be halted before all of the water-soluble glass is dissolved, thereby providing a body having a porous calcium phosphate layer overlying a water-soluble glass core. In addition, the process may be halted before the transformation of amorphous calcium phosphate to hydroxyapatite is complete; that is, the porous calcium phosphate layer may be substantially amorphous calcium phosphate, substantially hydroxyapatite, or a combination of the two.

By selection of the geometric configuration of the starting water-soluble glass body and by selection and control of the water-soluble glass dissolution, calcium phosphate deposition, and calcium phosphate transformation steps of the process of the present invention, calcium phosphate bodies having a variety of shapes, sizes and compositions may be obtained. In general, the initial geometric configuration and size of the starting water-soluble glass body determines the geometric configuration and size of the finished body since the calcium phosphate body is formed by the substantially uniform deposition of calcium phosphate onto the starting water-soluble glass body. For example, calcium phosphate bodies in the form of a disc, sphere, fiber, rod or virtually any shape or size into which glass may be molded or otherwise formed may be derived from starting glass bodies of the same approximate shape and size. Additionally, calcium phosphate bodies with an irregular shape may be prepared from water-soluble glass bodies prepared by crushing or shattering the glass. In addition, by controlling the dissolution, deposition and transformation, the resulting calcium phosphate body, in each of these various geometric shapes and sizes may optionally contain a core of glass, a void core (see, e.g., FIG. 1), amorphous calcium phosphate, hydroxyapatite and combinations thereof.

The size of the calcium phosphate body is influenced by the dissolution rate of the water-soluble glass body containing calcium and the solubility of the calcium phosphate produced. If the solubility of the calcium phosphate is low, the phosphate solution becomes supersaturated with calcium phosphate shortly after immersing the water-soluble glass body containing calcium in the phosphate solution, due to the reaction of the Ca²⁺ ions released from the dissolving water-soluble glass body with PO₄³⁻ and OH⁻ ions in solution. Upon supersaturation of the solution, calcium phosphate will precipitate on the outer surface of the undissolved water-soluble glass body where the undissolved water-soluble glass body has not significantly decreased in size. As the transformation process proceeds, the outer diameter of the calcium phosphate layer will remain substantially the same size as the water-soluble glass body. This phenomenon is due to the transformation occurring from the outside of the body to the inside as described above. However, if the dissolution rate of the water-soluble glass body is such that half of the water-soluble glass body has dissolved before the phosphate solution becomes supersaturated with calcium phosphate, then the size of the resulting phosphate body will be approximately half the size of the original water-soluble glass body. Thus, the relative rate difference of dissolution of the water-soluble glass body and the precipitation of the calcium phosphate layer approximately determines the relative size difference of the water-soluble glass body and the calcium phosphate body. Table 1 contains solubility product data for calcium phosphate compounds.

TABLE 1

| | | | Calcium |
|---|---|---|---|
| | Ionic | Solubility Product | concentration at |
| Salt | Composition | (25° C.) | Equilibrium (M) |
| Brushite | Ca(HPO₄)2H₂O | 2.32 × 10⁻⁷ | 4.8 × 10⁻⁴ |
| Tricalcium | Ca₃(PO₄)₂ | 2.83 × 10⁻³⁰ | 1.45 × 10⁻⁶ |
| phosphate | | | |
| (TCP) | | | |
| Octacalcium | Ca₄H(PO₄)₃ | 2 × 10⁻⁴⁹ | 1.08 × 10⁻⁶ |
| phosphate | | | |
| (OCP) | | | |
| Hydroxyapatite | Ca₅(PO₄)₃OH | 2.34 × 10⁻⁵⁹ | 4.36 × 10⁻⁷ |
| (HAp) | | | |
| Fluorapatite | Ca₅(PO₄)₃F | 3.16 × 10⁻⁶⁰ | 3.47 × 10⁻⁷ |
| (FA) | | | |

In addition, the solubility of the calcium phosphate compounds is affected by the pH of the solution used for dissolution. For example, hydroxyapatite is more soluble in acidic solution and less soluble in alkaline solution because the acid reacts with the hydroxide ions, thus reducing the hydroxide concentration in solution and causing the equilibrium between the solid and the ions in solution to shift toward the ions in solution; this phenomenon occurs in order to replace the hydroxide ions in solution lost upon reaction with the acid. The opposite shift occurs in alkaline solution, thus the equilibrium shifts toward the solid and the solubility of the hydroxyapatite is decreased.

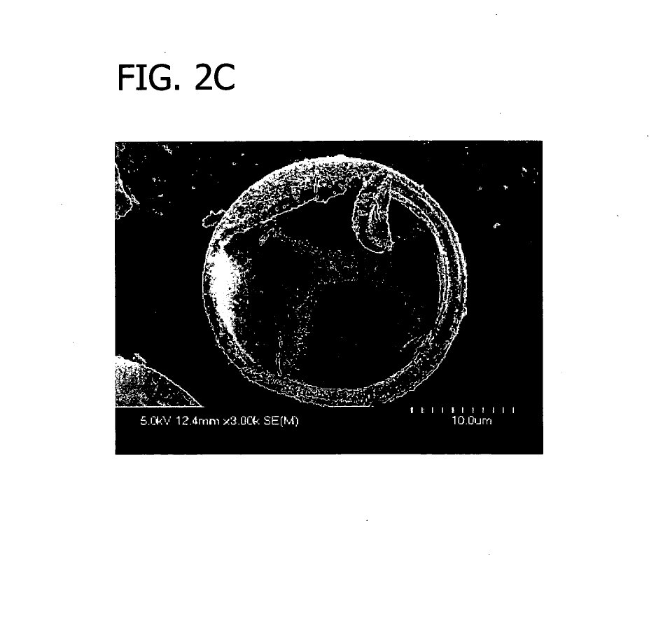

As illustrated in FIG. 1, the calcium phosphate body may be hollow and/or porous. In general, the size of the void depends on the amount of calcium in the glass and the shape of the starting soluble glass body. For example, when the water-soluble glass body is a sphere, the alkali metal oxide component is Li₂O and the glass former is B₂O₃, a CaO content of less than about 40 wt. % results in a hollow calcium phosphate sphere. Generally, decreasing amounts of calcium tend to increase the size of the hollow cavity within a calcium phosphate body. Without being bound by theory, it is presently believed that the calcium oxide in the water-soluble glass reacts with the phosphate solution to produce calcium phosphate, and thus, the amount of calcium in the water-soluble glass body limits the amount of calcium phosphate formed. As the amount of calcium oxide in the water-soluble glass increases, therefore, it is believed that more calcium phosphate is formed which can deposit on the surface of the body, thus increasing the wall thickness of calcium phosphate and decreasing the volume of the hollow cavity of the body to the point where there is no longer a hollow cavity in the body. The calcium phosphate microspheres shown in FIG. 2 were prepared from lithium-borate water soluble glasses containing 15 wt. % CaO, 10.7 wt. % Li₂O and 74.3 wt. % B₂O₃, which produces a hollow calcium phosphate body.

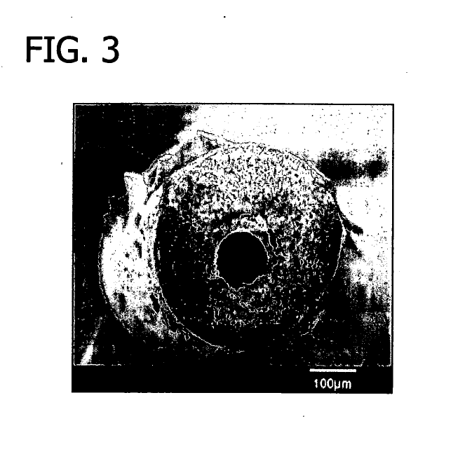

In another embodiment, the water-soluble glass body is hand drawn into fibers when the glass is in the molten state. Upon transformation of the fibers of the water-soluble glass with an aqueous phosphate solution, hollow or porous fibers of calcium phosphate form as shown in FIG. 3. In one embodiment in which the water-soluble glass bodies are drawn into fibers, the Ca compound is CaO and the glass formed is a glass containing 10-15 wt. % CaO; typically, the glass fiber contains 10 wt. % CaO and upon transformation of the water-soluble glass fiber to a calcium phosphate fiber, the fiber produced is hollow.

In a further embodiment, the calcium phosphate fiber formed by have an outer diameter (o.d.) of about 10 μm to about 10,000 μm and an inner diameter (i.d.) of about 1 μm to slightly less than about 10,000 μm. The length of the calcium phosphate fiber may be about 50 μm to about 50,000 μm. The aspect ratio (the ratio of the length to the o.d.) of the fibers may be about 5 to 1 to about 100,000 to 1; for example, the aspect ratio may typically be about 5:1 to about 100:1.

In yet another embodiment, the water-soluble glass body and the calcium phosphate body may be in the shape of a flake. The length and width of the flake may be about 50 μm to about 10,000 μm; the height of the flake is about 10 μm to about 2,000 μm. The aspect ratio of the gross length or the gross width to the height is about 5 to 1 to about 100 to 1. The shape defined by the longer dimensions of length and width may be circular, polygonal, ellipsoid, and the like and is not critical.

After the soluble glass body is transformed into a calcium phosphate body (to the desired extent) in the aqueous phosphate solution, the calcium phosphate body may be heat treated to modify the calcium phosphate layer. In general, heat treating the calcium phosphate body at a temperature in excess of 700° C. tends to convert any hydroxyapatite to tricalcium phosphate, Ca₃(PO₄)₂. However, hollow and porous particles may be heat treated to increase or decrease the permeability of the body. The resulting permeability or porosity will depend on the temperature and time of the heat treatment, where a longer treatment at a higher temperature causes the porosity of the calcium phosphate body to decrease. Thus, all the geometries or shapes of the calcium phosphate bodies may be heat treated as described above to produce calcium phosphate bodies with varying porosity.

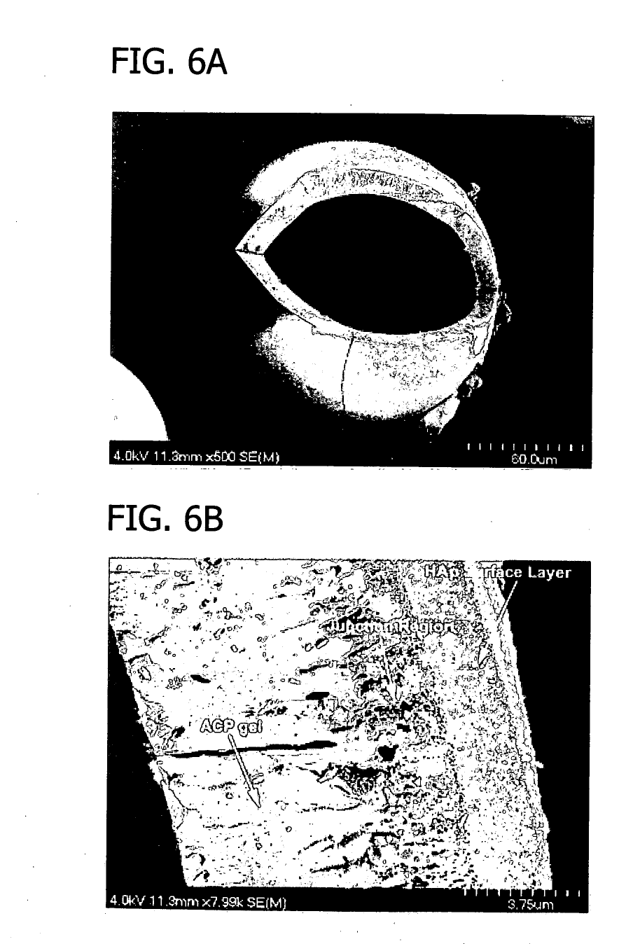

The calcium phosphate bodies of the present invention may be used in a variety of applications. In one embodiment, the hollow calcium phosphate microspheres are used as delivery systems for fluids which would benefit from a time released delivery method. The fluid to deliver may be a drug, vitamin, nutrient or the like. The calcium phosphate microspheres may be filled with the selected fluid by immersing the hollow calcium phosphate microspheres in the fluid and evacuating the gas from above the fluid to exchange the gas in the hollow microspheres for the fluid. The rate of release of the fluid may be manipulated by heat treating the calcium phosphate microspheres prior to filling with the fluid at various temperatures. The temperature of the heat treatment depends on the composition of the body and will be below the melting temperature of the substance. In one embodiment, the calcium phosphate body contains hydroxyapatite and may be heat treated at temperatures of 90° C., 100° C., 125° C., 200° C., 300° C., 400° C., 500° C., 600° C., 700° C., 800° C. or 900° C. for about 0.5 to about 48 hours. Particularly, the hydroxyapatite bodies will be heat treated for about 1 hour. Generally, as described above, the higher the heat treatment temperature for a constant time, the less permeable or porous the walls of the hollow microspheres and the slower the rate of fluid release. This use is described in more detail in Example 4. A graph of the release of bovine serum albumin from hollow hydroxyapatite microspheres is shown in FIG. 6.

In one embodiment, the hydroxyapatite spheres will be heat treated to provide a permeability that results in an initial rate of drug release in solution or in the body fluids of a subject that is about 0.1 to about 16 μg/mL·h and second rate of drug release in solution of about 0.01 to about 1 μg/mL·h. More particularly, the first rate of drug release will be about 1 to about 2 μg/mL·h and a second rate of drug release will be about 0.05 to about 0.1 μg/mL·h.

In another embodiment, the calcium phosphate bodies of the present invention may be used as a synthetic bone substitute. The calcium phosphate bodies may be produced in various sizes and shapes by immersing or contacting a water-soluble glass containing CaO with a phosphate solution. By increasing the CaO content in the water-soluble glass to 40 wt. %, calcium phosphate bodies with increased mechanical strength are produced. The increased mechanical strength may be desirable for use as a synthetic bone substitute. In a further embodiment, the (i) water-soluble glass body may be doped with cations or anions and/or (ii) cations or anions may be added to the phosphate solution to provide doped calcium phosphate bodies as described above of optimum composition for use as a synthetic bone substitute. In particular, when the water-soluble glass body contains CaO, doping the phosphate solution with CO₃²⁻ ions provides a calcium phosphate body with a composition that closely mimics the composition of natural bone. In a further embodiment, calcium phosphate bodies may be used as bone filler or dental implants.

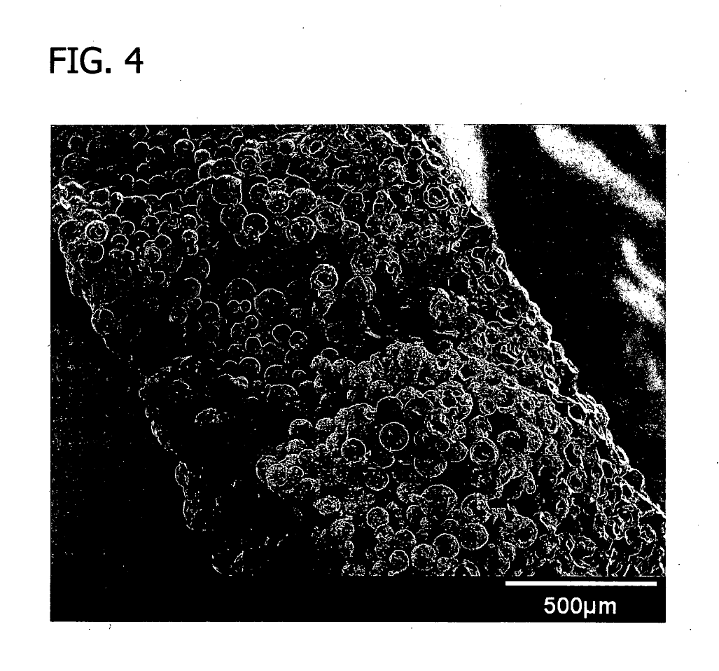

In a further embodiment, calcium phosphate bodies of the present invention may be used as separation media. In this embodiment, calcium phosphate bodies can be agglomerated into larger objects during the reaction of the water-soluble glass with the aqueous phosphate solution (as described in Example 3) or the reacted particles may be aggregated together by heat treating. The aggregates or agglomerates (as shown in FIG. 4) may be fabricated into any desired shape and used as ion exchange media or filtering agents. The shape and size of the agglomerates of calcium phosphate bodies are dependent on the number of water-soluble glass bodies to be transformed into calcium phosphate bodies, the shape and size of the reaction vessel and the degree of agitation of the phosphate solution while the transformation process is occurring. In one embodiment, the agglomerate contains at least about 10 calcium phosphate bodies. In another embodiment, generally, the water-soluble glass bodies fill the bottom of a container, are not agitated significantly and are immersed in or contacted with phosphate solution. In this embodiment, the shape and size of the agglomerate produced upon transformation of the water-soluble glass bodies into calcium phosphate bodies depends on the depth of the water-soluble glass bodies in the container. If the depth of the glass bodies is small, agglomerates in the shape of a disc will be formed. If the depth of the glass bodies is larger, agglomerates in the shape of a cylinder, similar to the agglomerate shown in FIG. 4, will be formed.

Optionally, agglomerates may be formed by heat treating the water-soluble glass bodies to sinter them into a plurality of water-soluble glass bodies prior to transformation in a phosphate solution. After sintering the glass bodies, the agglomerate of glass bodies would be contacted with a phosphate solution and allowed to transform into an agglomeration of calcium phosphate bodies.

In still a further embodiment, macroscopically smooth bodies of calcium phosphate are used as a separation media for affinity chromatography. Macroscopically smooth bodies appear substantially smooth to the naked eye. When bodies are too small to observe without magnification, macroscopically smooth bodies appear substantially smooth at magnifications up to about 200×. Macroscopically smooth bodies such as spheres may have a diameter of about 0.5 μm to about 90 μm. Macroscopically smooth bodies which are fibers may be of the dimensions described above. Macroscopically smooth bodies of ellipsoids may have a shorter diameter of about 0.5 μm to about 500 μm and a longer diameter of about 1 μm to about 600 μm. The calcium phosphate bodies can adsorb and desorb proteins, nucleic acids, polypeptides, biological products and the like, to effect separation and purification of these species. The calcium phosphate bodies may be packed into a column or bed or swirled in a container with the chemical species contained in the fluid to be separated. The fluid to be separated may be contacted with the calcium phosphate bodies once or in a continuous loop. In one embodiment, hydroxyapatite crystals have positively charged adsorption sites formed by calcium ions and negatively charged adsorption sites formed by oxygen ions, which are members of phosphate (PO₄³⁻) ions. Thus, the surface of the hydroxyapatite crystals have a plurality of positive and negative adsorption sites.

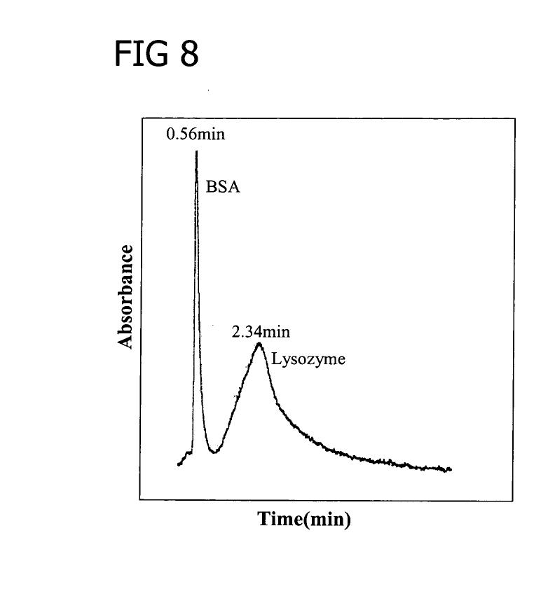

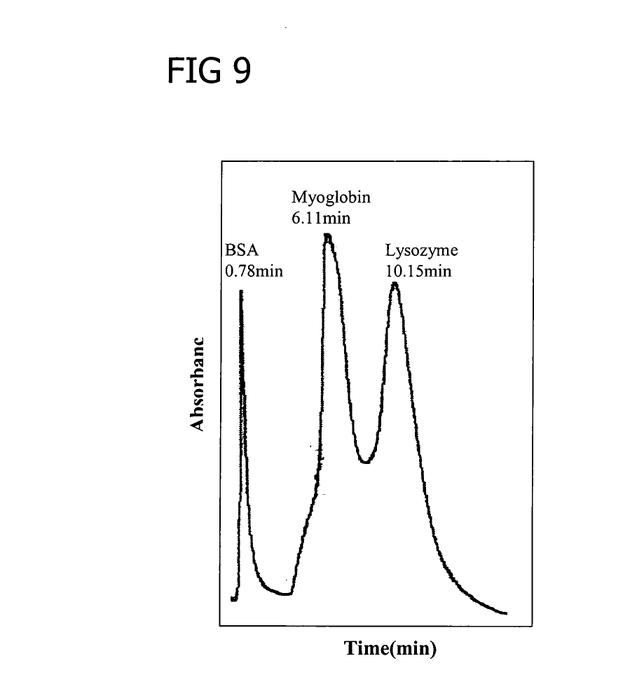

Without being bound by theory, generally, liquid chromatography operates to separate components of a sample by differentiating between the components on the basis of the relative affinities for the stationary phase (here, hydroxyapatite microspheres) and the mobile phase (here, the phosphate solutions). If the component's affinity for the stationary phase is high, the component will take longer to elute than a component whose affinity for the stationary phase is low. In Example 5, the isoelectric point of the proteins was a distinguishing property to predict whether the protein component would have a high affinity for the hydroxyapatite stationary phase. In this case, a higher isoelectric point for the protein meant a stronger affinity of that protein for the hydroxyapatite stationary phase. In one embodiment, the calcium phosphate bodies used as the stationary phase have a specific surface area of about 50 to about 400 m²/g. HPLC chromatograms showing the separation of proteins on a column packed with hydroxyapatite microspheres are shown in FIGS. 8 and 9 and described in more detail in Example 5.

In still another embodiment, calcium phosphate bodies may be used for diagnostic imaging. Hollow or porous spheres formed in the present invention may be doped with the desired ion for the particular diagnostic imaging application by doping the initial glass with a metal oxide. In a further embodiment, hollow calcium phosphate bodies may have the porosity of the calcium phosphate reduced by heat treatment above 700° C. and be filled with a gas useful for diagnostic imaging where the lower porosity will reduce the rate of gas release from the inside of the hollow calcium phosphate body. In addition, hollow calcium phosphate bodies may be used for storage of gases by filling the bodies with a gas, heat treating the body to reduce or eliminate the permeability of the body walls and storing the gas-filled calcium phosphate bodies.

Accordingly, one aspect of the present invention is a calcium phosphate body in the form of a hollow fiber or a hollow or porous sphere with a diameter of less than about 1 μm. Another aspect of the present invention is a calcium phosphate body comprising amorphous calcium phosphate; for example, amorphous calcium phosphate bodies may be composed of tricalcium phosphate (TCP) or octacalcium phosphate (OCP) or mixtures thereof. Another aspect of the present invention is a calcium phosphate body which comprises hydroxyapatite. Another aspect of the present invention is a calcium phosphate body that is hollow or porous. The porosity of the calcium phosphate bodies can be characterized by a pore size of about 20 nm to about 3 μm.

### Definitions

All composition percentages are understood to be weight percentages and are calculated by the formula (weight of component/weight of total composition)×100%.

Unless defined otherwise, an “alkali metal” is an element other than hydrogen that is located in Group 1A of the periodic table. Exemplary alkali metals are lithium, sodium, potassium, rubidium, cesium and francium.

Unless defined otherwise, an “alkaline earth metal” is an element found in Group 2A of the periodic table. Exemplary alkaline earth metals are beryllium, magnesium, calcium, strontium and barium.

Unless defined otherwise, a “dopant” is a material that is substituted for other ions in the calcium phosphate body.

Unless defined otherwise, “supersaturation” means the concentration of the species in solution is greater than the solubility of that species.

The following examples illustrate the invention.

### EXAMPLES

### Example 1

### Water-Soluble Glasses Containing Calcium

The desired amounts of CaCO₃₍ₛ₎, CaSO₄₍ₛ₎ or a combination of CaCO₃₍ₛ₎ CaSO₄₍ₛ₎, were mixed with carbonates of alkali metals and a glass former to make a specific glass composition. This mixture was melted in a platinum/rhodium crucible at about 1050° C. for approximately 30 minutes and then quenched between two cold stainless steel plates to prevent crystallization, thus forming the water-soluble glass body containing calcium. A combination of CaCO₃₍ₛ₎ and CaSO₄₍ₛ₎ was used when the wt. % of CaO was greater than 25%. The following glass compositions in Tables 2 and 3 were made using the above procedure.

TABLE 2

| CaO (wt. %) | Li₂O (wt. %) | B₂O₃ (wt. %) |
|---|---|---|
| 5 | 12.0 | 83.0 |
| 10 | 11.3 | 78.7 |
| 15 | 10.7 | 74.3 |
| 25 | 9.4 | 65.6 |
| 40 | 7.5 | 52.5 |
| 50 | 6.3 | 43.7 |

TABLE 3

| Glass | CaO (wt. %) | Na₂O (wt. %) | B₂O₃ (wt. %) |
|---|---|---|---|
| 1-2-6 | 9.3 | 20.7 | 70.0 |
| 2-2-6 | 17.1 | 18.9 | 64.0 |

### Example 2

### Water-Soluble Glass Bodies

Microspheres of the water-soluble glass were prepared by dropping crushed glass (frit) through a small dense ceramic tube (0.25″ ID) into a vertical tube furnace containing a larger dense ceramic tube (3.25″ ID) heated to a maximum temperature of 1000° C. to 1100° C. for the glasses containing up to 40 wt. % CaO. The frit was dropped into the small tube using a vibrating spatula and exited the tube just above the hot zone of the furnace. After falling through the furnace, where the particles melted and became spherical, the microspheres were collected in a glass jar attached to the bottom of the large tube and sieved into various size ranges using both dry and wet sieving with acetone. FIG. 2 shows scanning electron microscope images of calcium phosphate bodies made by reacting a water-soluble glass body with a 0.25M K₂HPO₄ solution at 37° C. for 24 hours. FIG. 2 shows calcium phosphate microspheres made from water-soluble glass bodies with the composition of 15 wt. % CaO, 10.7 wt. % Li₂O and 74.3 wt. % B₂O₃.

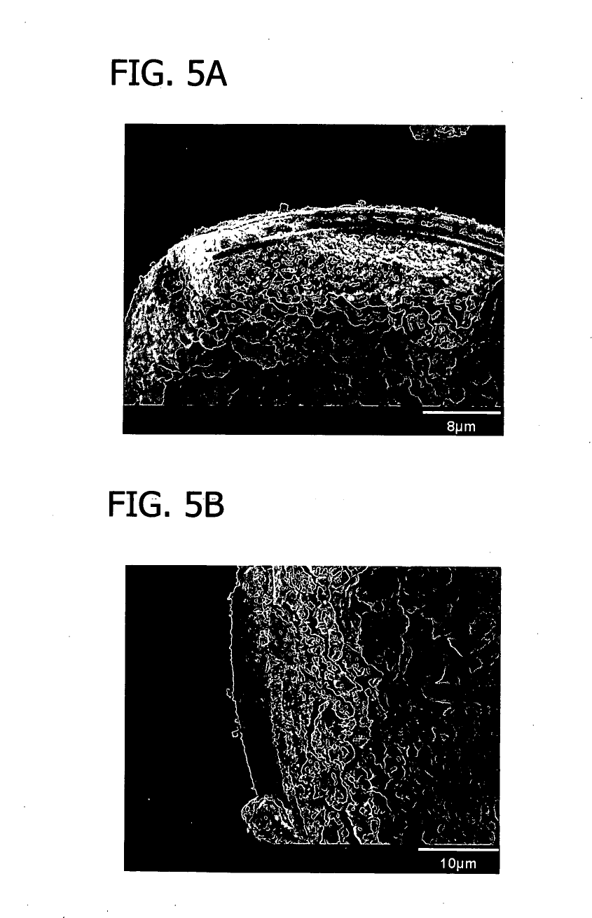

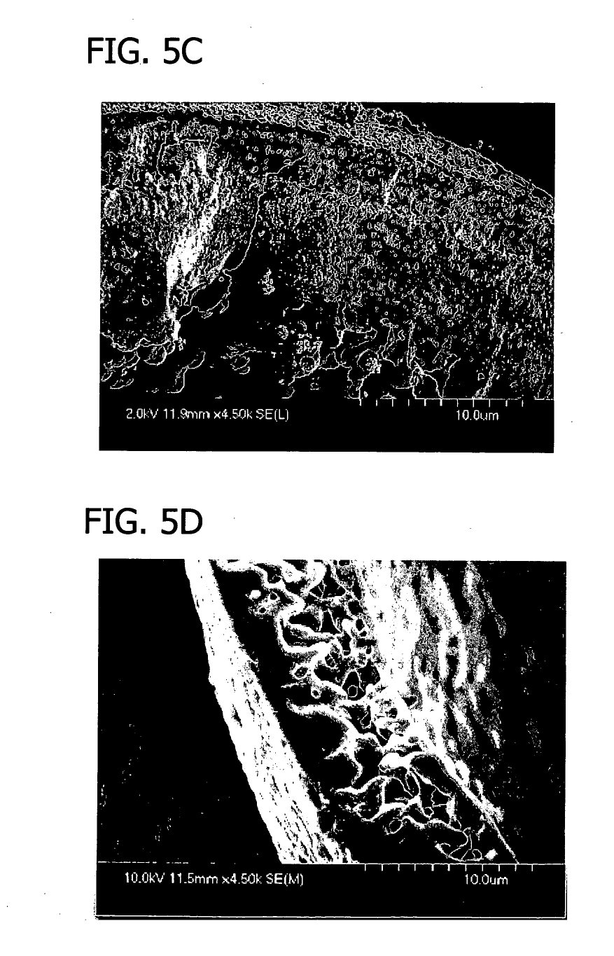

Hollow calcium phosphate microspheres were made by transforming a water-soluble glass body containing 15 wt. % CaO, 10.7 wt. % Li₂O and 74.3 wt. % B₂O₃ into a calcium phosphate body by immersing the water-soluble glass body in a 0.25M K₂HPO₄ solution at 37° C. for 24 hours. Subsequently, the calcium phosphate bodies were heat treated for one hour at (a) 300° C., (b) 500° C., (c) 600° C. and (d) 900° C.; the scanning electron microscope images are shown in FIG. 5.

Glass fibers were made by hand drawing the glass from the melt. Hollow calcium phosphate fibers were made by transforming water-soluble glass bodies containing 10 wt. % CaO, 11.3 wt. % Li₂O and 87.7 wt. % B₂O₃ in a 0.25 M K₂HPO₄ solution at 37° C. for 3 days. A scanning electron microscope image of a hollow calcium phosphate fiber is shown in FIG. 3.

Other shapes are made by casting the glass into graphite molds. For example, bars and rods are made by casting the molten glass into graphite molds.

### Example 3

### Agglomerates of Calcium Phosphate Bodies

Agglomerates of the calcium phosphate bodies were produced by allowing the individual particles of the water-soluble glass to bond together to form aggregates or agglomerates of calcium phosphate bodies upon transformation in a 0.25M K₂HPO₄ solution. Discs composed of calcium phosphate bodies were made by transforming a loose layer, about 1 mm thick, of glass microspheres (containing 15 wt. % CaO, 10.7 wt. % Li₂O and 74.3 wt. % B₂O₃ and having a diameter of 106 to 150 μm) for 3 days in a 0.25M K₂HPO₄ solution (pH 9.0) at 37° C. In another experiment, a layer of glass microspheres with the same composition as above was much thicker and formed an agglomerate that had a cylindrical shape. This cylindrical agglomerate is shown in a scanning electron microscope image in FIG. 4.

Alternately, with vigorous mixing of the solution during the transformation of the water-soluble glass bodies containing calcium to calcium phosphate bodies by immersing the water-soluble glass bodies in 0.25M K₂HPO₄ solution.

### Example 4

### Drug Delivery Vehicles

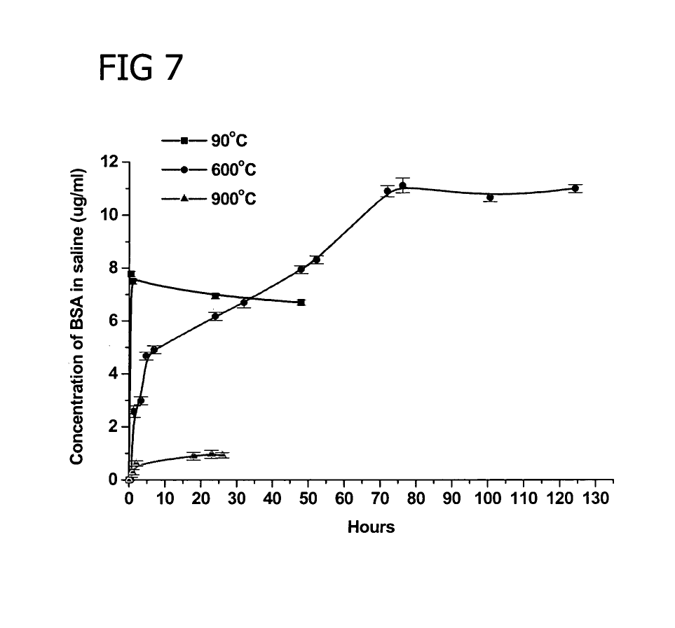

Hollow hydroxyapatite microspheres were fabricated by transforming water-soluble glass bodies containing calcium into calcium phosphate bodies by immersing the water-soluble glass bodies in a 0.25M K₂HPO₄ solution. Preferably, the water-soluble glass bodies contained 10-15 wt. % CaO. The hollow calcium phosphate microspheres were filled with a drug solution by immersing the calcium phosphate microspheres in a solution containing the drug and evacuating the gas above the solution until the gas inside the hollow calcium phosphate microspheres was replaced by the drug solution. This exchange was complete when the release of gas bubbles from the calcium phosphate microspheres ceased. FIG. 7 shows a graph of the concentration of bovine serum albumin (BSA) measured in a saline solution at 37° C. as a function of time after two grams of calcium phosphate microspheres (106 to 150 μm in diameter) filled with BSA solution were placed in 300 mL of saline. The three curves correspond to hollow microspheres made by reacting a water-soluble glass containing 15 wt. % CaO, 10.7 wt. % Li₂O and 74.3 wt. % B₂O₃ in a 0.25 M K₂HPO₄ solution at 37° C. for 24 hours, then heat treating at 90° C., 600° C. and 900° C. for and hour. After heat treating, the hollow microspheres were filled with a 3.5 mg/ml BSA saline solution. The data were normalized to the first data point.

Specific surface areas (m²/g) of calcium phosphate spheres were measured. The water-soluble glass spheres (15 wt. % CaO, 10.7 wt. % Li₂O and 74.3 wt. % B₂O₃) were transformed to calcium phosphate spheres by immersing the water-soluble glass spheres in a 0.25M K₂HPO₄ solution for 24 hours at 37° C. Subsequently, the spherical particles were fired at 125° C., 300° C., 500° C., 600° C. and 700° C. for one hour. The results of this analysis are listed in Table 4.

TABLE 4

| Firing Temperature (° C.) | Specific Surface Area (m²/g) |
|---|---|
| 125 | 147.6 |
| 300 | 47.9 |
| 500 | 27.3 |
| 600 | 13.5 |
| 700 | 9.9 |

### Example 5

### Separation Media

Hydroxyapatite bodies formed by the process detailed above were used as separation media. Two water-soluble glasses (compositions are listed in Table 3 above) were made by the above process. The glass was crushed with a steel anvil and crusher and sieved to size ≦90 μm. A propane torch was used for spheroidizaton. Glass particles were passed through a propane flame, where the particles melted and became spherical. The water-soluble glass microspheres were sieved into two groups, 45-90 μm and <45 μm. Both groups were immersed in a 0.25M K₂HPO₄ solution at 37° C. and a pH of 9.0±0.05 for 24 hours to form hydroxyapatite bodies. The reaction solution was vigorously stirred to prevent clumping or settling of the bodies. After reaction, the hydroxyapatite bodies were rinsed with distilled water, then rinsed with ethanol and dried in air at room temperature for 12 hours.

Chicken egg lysozyme (Sigma Co. Cat. #L-6876) was used to evaluate the protein adsorption of the reacted glass particles and microspheres. An absorbance vs. concentration curve was produced to calibrate the UV detector response to the concentration of the chicken egg lysozyme. Elution chromatography was carried out using a phosphate eluent on a sequence-programmable HP series 1050 HPLC instrument. A 5 mL sample of the protein mixture was injected into the HPLC system and the sample elution was monitored by measuring the UV absorption at a wavelength of 280 nm. The chromatograms were recorded with an automatic HP 339 series 2 integrator.

The proteins used were bovine serum albumin (BSA, Sigma Co. Cat. # A-7906), chicken egg lysozyme (Sigma Co. Cat. #L-6876) and horse heart myoglobin (Nutrational Biochemical Co., Cat. # 2920). Each component was dissolved in deionized water; the initial concentration of each protein was 10 mg/mL which was diluted to 0.5 mg/mL with deionized water.

Hydroxyapatite microspheres (45-90 μm) made from 2-2-6 water-soluble glass from Table 3 were packed into a steel column used for the binary protein mixture separation and microspheres smaller than 45 μm were used for the ternary protein mixture separation. The steel column (4.6 mm I.D., 80 mm length) was packed with the hydroxyapatite microspheres made from 2-2-6 water-soluble glass by hand. A metal filter was mounted to each end of the column to avoid contamination and both the column and the filters were washed in an ultrasonic water bath before packing. Approximately 1.5 g of dry hydroxyapatite microspheres were poured into the column using a small funnel. The column side wall was tapped gently to help the microspheres pack evenly.

The HPLC instrument was equipped with two solvent reservoirs. Before the solvents entered the HPLC system, they passed through degassers. The solvents were mixed in the mixing vessel to achieve the desired phosphate concentration.

The packed column was washed overnight with deionized water at a flow rate of 0.01 mL/min. The phosphate eluent was then introduced and the flow rate was gradually increased to 1.0 mL/min. The sample was introduced into the column through a sample loop, wherein the sample injector injected the sample into the effluent upon filling of the sample loop. A UV-Vis spectrometer was used to measure the absorbance of the effluent in a flow-through cell. The measured absorbance data was plotted by an integrator as a function of time. HPLC chromatograms shown in FIGS. 8 and 9 and the following Tables illustrate the separation of the proteins using the hydroxyapatite microspheres as the column packing material.

Table 5 shows the specific surface areas of the calcium phosphate bodies transformed from 1-2-6 and 2-2-6 irregular glass particles and 2-2-6 glass microspheres. Table 6 shows the retention time and time intervals between the BSA and lysozyme peaks for the isocratic elution of the binary protein mixture. Where solution A is a 0.01M phosphate solution and solution B is a 0.1M phosphate solution. Table 7 shows the gradient elution time schedules used for the binary protein mixture of BSA and lyxozyme, where solution A and solution B are defined above.

Table 8 shows the retention time and time intervals in minutes between the BSA and lysozyme peak for the gradient elutions. Table 9 shows the gradient elution time schedule used for the ternary protein mixture where solution A is defined above and solution C is a 0.5M phosphate solution. Table 10 shows the isoelectric points of BSA, myoglobin and lysozyme. FIGS. 8 and 9 are HPLC chromatograms illustrating the separation of BSA, myoglobin and lysozyme using hydroxyapatite microspheres as described herein above.

TABLE 5

| Transformed | | | |
|---|---|---|---|
| Glass Body | 1-2-6 Particles | 2-2-6 Particles | 2-2-6 microspheres |
| Specific surface | 52.3 | 68.7 | 190.4 |
| area (m²/g) | | | |

TABLE 6

| | | | | | | | | |
|---|---|---|---|---|---|---|---|---|
| Solution A (%) | 10 | 20 | 30 | 40 | 50 | 60 | 70 | 75 |
| Solution B (%) | 90 | 80 | 70 | 60 | 50 | 40 | 30 | 25 |
| BSA retention | 0.81 | 0.80 | 0.80 | 0.81 | 0.83 | 0.90 | 0.94 | 1.08 |
| time (min) | | | | | | | | |
| Lysozyme | 1.18 | 1.23 | 1.36 | 1.53 | 1.58 | 2.18 | | |
| retention | | | | | | | | |
| time (min) | | | | | | | | |
| Time intervals | 0.37 | 0.43 | 0.56 | 0.72 | 0.75 | 1.28 | | |
| (min) | | | | | | | | |

TABLE 7

| Gradient No. | Time (min) | Solution A (%) | Solution B (%) |
|---|---|---|---|
| Gradient 1 | 0 | 75 | 25 |
| | 2 | 75 | 25 |
| | 3 | 0 | 100 |
| Gradient 2 | 0 | 75 | 25 |
| | 1 | 75 | 25 |
| | 2 | 0 | 100 |
| Gradient 3 | 0 | 70 | 30 |
| | 1 | 70 | 30 |
| | 2 | 0 | 100 |
| Gradient 4 | 0 | 70 | 30 |
| | 0.5 | 70 | 30 |
| | 1 | 0 | 100 |
| Gradient 5 | 0 | 70 | 30 |
| | 0.2 | 70 | 30 |
| | 0.5 | 0 | 100 |

TABLE 8

Retention times

| Gradient No. | BSA | Lysozyme | Time Intervals |
|---|---|---|---|
| 1 | 1.05 | 6.43 | 5.38 |
| 2 | 0.95 | 6.46 | 5.51 |
| 3 | 0.83 | 5.27 | 4.44 |
| 4 | 0.85 | 4.27 | 3.42 |
| 5 | 0.83 | 3.47 | 2.64 |

TABLE 9

| | Time (min) | | |
|---|---|---|---|
| | 0 | 5 | 5.5 |
| Solution A (%) | 50 | 50 | 0 |
| Solution C (%) | 50 | 50 | 100 |

TABLE 10

| Proteins | BSA | Myoglobin | Lysozyme |
|---|---|---|---|
| Isoelectric point | 4.7 | 7 | 10.5-11 |

In view of the above, it will be seen that the several objects of the invention are achieved and other advantageous results attained.

As various changes could be made in the above methods without departing from the scope of the invention, it is intended that all matter contained in the above description or shown in the accompanying drawings shall be interpreted as illustrative and not in a limiting sense.

What is claimed is:

1. A calcium phosphate body wherein the body is an calcium phosphate agglomerate, the agglomerate being a product of an agglomeration as a plurality of water-soluble glass bodies is transformed into a plurality of calcium phosphate bodies.

2. The agglomerate of claim 1 wherein the agglomerate contains at least about 10 calcium phosphate bodies.

3. The agglomerate of claim 1 wherein the agglomerate has a width of at least about 1 μm.

4. The calcium phosphate body of claim 1 wherein the body is an calcium phosphate agglomerate, the agglomerate being a product of an agglomeration as a plurality of water-soluble glass bodies is sintered into an agglomerate and subsequently transformed into an agglomerate of calcium phosphate bodies.

5. The calcium phosphate body of claim 1 wherein the body is hollow.

6. The calcium phosphate body of claim 1 wherein the body is porous.

7. The calcium phosphate body of claim 1 wherein the body is hollow and porous.

8. A process for making a calcium phosphate body comprising contacting a water-soluble glass body in the form of a sphere with a diameter of less than about 1 μm, fiber, flake or ellipsoid and a phosphate solution wherein the water-soluble glass body contains about 1 to about 40 wt. % CaO, about 5 to about 65 wt. % alkali metal oxide component and about 20 to about 94 wt. % of a glass former.

9. The process of claim 8 wherein the water-soluble glass body contains about 15 wt. % of CaO.

10. The process of claim 8 wherein the alkali metal oxide component is Li₂O, Na₂O, K₂O, Rb₂O, Cs₂O or mixtures thereof.

11. The process of claim 8 wherein the alkali metal oxide is Li₂O.

12. The process of claim 8 wherein the water-soluble glass body contains about 10 to about 15 wt. % CaO and about 8 to about 15 wt. % of the alkali metal oxide wherein the alkali metal oxide is Li₂O.

13. The process of claim 8 wherein the glass former is B₂O₃.

14. The process of claim 8 wherein the water-soluble glass body contains about 10 to about 15 wt. % CaO and about 8 to about 15 wt. % of the alkali metal oxide wherein the alkali metal oxide is Li₂O, and containing about 70 to about 82 wt. % of B₂O₃.

15. The process of claim 8 wherein the calcium phosphate is amorphous calcium phosphate or hydroxyapatite.

16. The process of claim 8 wherein the water-soluble glass body and the phosphate solution are contacted for a time ranging from about 1 hour to 2 weeks.

17. The process of claim 8 wherein the water-soluble glass body and the phosphate solution are contacted for a time ranging from about 4 hours to 24 hours.

18. The process of claim 8 wherein the water-soluble glass body and the phosphate solution are contacted at a temperature of about 20° C. to about 90° C.

19. The process of claim 8 wherein the water-soluble glass body and the phosphate solution are contacted at a temperature of about 37° C.

20. The process of claim 8 wherein the phosphate solution has a pH of about 7 to about 10.

21. The process of claim 8 wherein the phosphate solution has a pH of about 9.

22. The process of claim 8 wherein the phosphate solution has a concentration of about 0.001 M to 1.0M.

23. The process of claim 8 wherein the phosphate solution has a concentration of about 0.25M.

24. The process of claim 8 wherein the water-soluble glass body and the phosphate solution are contacted at a temperature of about 37° C. for a time of about 24 hours and the phosphate solution has a concentration of about 0.25M and a pH of about 9.

25. A process for making an agglomerate of calcium phosphate bodies comprising contacting a plurality of water-soluble glass bodies with a phosphate solution and allowing the calcium phosphate bodies to fuse together as the water-soluble glass bodies are transformed to calcium phosphate bodies.

26. The process of claim 25 wherein the water-soluble glass bodies contain about 1 to about 40 wt. % CaO, about 5 to about 65 wt. % alkali metal oxide component and about 20 to about 94 wt. % of a glass former.

27. A method for delivering a fluid comprising (a) heat treating a calcium phosphate body at a temperature between about 30° C. and about 1500° C., (b) filling the calcium phosphate body with the fluid and (c) administering the calcium phosphate body filled with the fluid to a subject.

28. The method of claim 27 wherein the calcium phosphate body is heat treated at about 90° C. to about 900° C.

29. The method of claim 27 wherein the calcium phosphate body is heat treated for about 0.5 hours to about 48 hours.

30. The method of claim 27 wherein a first rate of fluid release in a body of the subject is about 0.1 to about 16 μg/mL·h.

31. The method of claim 27 wherein a second rate of fluid release in a body of the subject is about 0.01 to about 1 μg/mL·h.

32. A method for separating a chemical species from a fluid containing the species by affinity chromatography comprising (a) contacting the fluid with a macroscopically smooth calcium phosphate body to adsorb the chemical species onto the calcium phosphate body, (b) separating the fluid from the calcium phosphate body and (c) desorbing the species from the calcium phosphate body.

33. The method of claim 32 wherein the calcium phosphate bodies are packed into a chromatography column.

34. The method of claim 32 wherein the calcium phosphate bodies are packed into a high performance liquid chromatography column.

35. The method of claim 32 wherein the calcium phosphate bodies are packed into a gas chromatography column.

36. The method of claim 32 wherein the calcium phosphate bodies are packed into a liquid chromatography column.

37. The method of claim 32 wherein the calcium phosphate bodies are packed into a bed.

38. The method of claim 32 wherein the calcium phosphate bodies are swirled in a vessel with the fluid.

39. The method of claim 32 wherein the calcium phosphate bodies are packed into a column or bed and the fluid is passed through the column or bed once.

40. The method of claim 32 wherein the calcium phosphate bodies are packed into a column or bed and the fluid is passed through the column or bed in a continuous loop.

41. The method of claim 32 wherein the calcium phosphate body is a hydroxyapatite sphere.

42. The method of claim 41 wherein the hydroxyapatite sphere has a diameter of about 0.5 μm to about 90 μm.

43. The method of claim 41 wherein the hydroxyapatite sphere has a diameter of about 45 μm to about 90 μm.

44. The method of claim 41 wherein the hydroxyapatite sphere has a diameter of about 0.5 μm to about 45 μm.

45. The method of claim 32 wherein the calcium phosphate body is prepared by contacting a water-soluble glass body and a phosphate solution wherein the water-soluble glass body contains about 9 to about 20 wt. % CaO, about 15 to about 25 wt. % Na₂O and about 55 to about 76 wt. % B₂O₃.

46. The method of claim 45 wherein the water-soluble glass body contains about 9.3 wt. % CaO, about 20.7 wt. % Na₂O and about 70.0 wt. % B₂O₃.

47. The method of claim 45 wherein the water-soluble glass body contains about 17.1 wt. % CaO, about 18.9 wt. % Na₂O and about 64.0 wt. % B₂O₃.

48. The method of claim 32 wherein the chemical species to be separated is a protein, a nucleic acid, a polypeptide or biological products.

49. The method of claim 48 wherein the protein is BSA, lysozyme, or myoglobin.

50. The method of claim 32 wherein the calcium phosphate body has a specific surface area of about 50 to about 400 m²/g.

51. A method for use of a calcium phosphate body as a bone substitute comprising administering the calcium phosphate body to a subject.

52. The method of claim 51 wherein the calcium phosphate body contains a dopant of CO₃²⁻.

53. A method for use of a calcium phosphate body as a diagnostic imaging agent comprising (a) filling the calcium phosphate body with a gas, (b) heat treating the calcium phosphate body and (c) administering the gas filled calcium phosphate body to a subject.

54. The method of claim 53 wherein the gas is air, hydrogen, nitrogen, oxygen, helium, neon, argon or xenon.

55. A regular or irregular particle of hydroxyapatite prepared from molded or crushed water-soluble glass, other than glass containing 20-35 wt% CaO, 20-35 wt. % Na₂O, 0-10 wt. % P₂O₅ and 30-50 wt. % B₂O₃, transformed in a phosphate solution at a temperature of less than about 100° C.

56. The particle of claim 55 wherein the water-soluble glass has a ratio of B₂O₃ to alkali metal oxide component of about 2.5 to 1 to about 3.5 to 1.

57. The particle of claim 55 wherein the water-soluble glass has a ratio of B₂O₃ to alkali metal oxide component of about 2.5 to 1 to about 3.5 to 1.
